(19) United States
(12) Patent Application Publication
(10) Pub. No.: US 2026/0115036 A1
(43) Pub. Date: Apr. 30, 2026

(54) METHOD, DEVICE, SYSTEM AND RECORDING MEDIUM FOR MANUFACTURING CUSTOMIZED ORAL DEVICE FOR SLEEP DISORDER IMPROVEMENT USING 3D MODELING

(71) Applicant: THE SLEEP FACTORY INC., Incheon (KR)

(72) Inventors: Junhyuk PARK, Incheon (KR); Dongwoo PARK, Incheon (KR)

(73) Assignee: THE SLEEP FACTORY INC., Incheon (KR)

(21) Appl. No.: 19/430,615

(22) Filed: Dec. 23, 2025

Related U.S. Application Data
(63) Continuation of application No. PCT/KR2025/015814, filed on Oct. 2, 2025.

(30) Foreign Application Priority Data
Oct. 8, 2024 (KR) 10-2024-0136178

(51) Int. Cl. A61F 5/56

(52) U.S. Cl. CPC A61F 5/566; A61F 2005/563

(57) ABSTRACT

A method of manufacturing a customized oral device for sleep disorder improvement using 3D modeling according to various embodiments of the present disclosure is disclosed. The method may include generating a tooth model including a maxillary model and a mandibular model, generating contour information based on the tooth model, generating a plurality of part models based on the contour information, and generating an oral device model by integrating the plurality of part models.

### CROSS-REFERENCE TO RELATED APPLICATION

This application is a continuation of International Application No. PCT/KR2025/015814, filed on Oct. 2, 2025, which claims priority to and the benefit of Korean Patent Application No. 10-2024-0136178, filed on Oct. 8, 2024, the disclosure of which is incorporated herein by reference in its entirety.

### BACKGROUND

### 1. Field of the Invention

Various embodiments of the present disclosure relate to a method of providing a user-customized oral device, and more particularly, to a method of manufacturing a customized oral device for sleep disorder improvement using 3D modeling.

### 2. Discussion of Related Art

Sleep is essential to human health and well-being. In particular, deep sleep is a critical time for physical recovery and mental stability. However, sleep disorders are becoming increasingly common in modern society. Among those sleep disorders, snoring and sleep apnea have a serious impact on many people. These sleep disorders may not only reduce sleep quality but may also lead to various health problems in the long term, such as cardiovascular disease, high blood pressure, and diabetes.

Snoring and sleep apnea are primarily caused by a narrowed airway. When airway stenosis obstructs the smooth flow of air, recurrent hypoxia occurs during sleep. This condition may lead to shallower sleep depth, resulting in persistent fatigue and daytime drowsiness. Furthermore, when hypoxia persists over a long period, it may lead to serious complications.

There are two major existing methods for treating sleep disorders. The first method uses continuous positive airway pressure (CPAP) to forcibly push air into the narrowed airway. This method is effective in preventing sleep apnea by continuously supplying air to maintain the airway and prevent the airway from collapsing. However, this method requires a user to continuously wear a device, which often leads to many users finding it uncomfortable for long-term use. Furthermore, the device requires maintenance and repeated hospital visits, imposing a financial and time burden on a patient.

The second method utilizes an oral device that a user can wear during sleep. This method is a non-invasive approach that secures an airway and induces smooth breathing with an oral device worn during sleep. The oral device may be used without surgery and is widely used as a relatively simple and safe method. However, the existing oral device is typically manufactured in a standardized form, and therefore may not perfectly fit oral structures and tooth conditions of individual users. As a result, there is concern that this may cause discomfort or limit the therapeutic effectiveness.

Accordingly, continuous efforts are being made to provide user-customized oral devices. However, the production of customized oral devices requires high technical expertise, which in turn increases costs. That is, due to the high costs and complex manufacturing process, many users find it difficult to access customized oral devices easily. Due to these issues, the need for technologies capable of efficiently designing and manufacturing customized devices tailored to individual users continues to grow.

### SUMMARY OF THE INVENTION

The present disclosure is directed to providing a method through which a user-customized oral device can be efficiently and economically designed and manufactured using 3D modeling.

Objects of the present disclosure are not limited to the above-described objects. That is, other objects that are not mentioned may be obviously understood by those skilled in the art from the following description.

To solve the above-described problem, a method of manufacturing a customized oral device for sleep disorder improvement using 3D modeling according to an aspect of the present disclosure is disclosed. The method may include generating a tooth model including a maxillary model and a mandibular model, generating contour information based on the tooth model, generating a plurality of part models based on the contour information, and generating an oral device model by integrating the plurality of part models.

The generating of the contour information may include generating a master model by applying a clearance distance corresponding to each of multiple regions of the tooth model, generating basic contour information based on the master model, and generating the contour information by correcting the master model based on the basic contour information.

The clearance distance may be set to allow for pressure distribution and a predetermined amount of movement, and determined differently for each region based on anatomical characteristics of a tooth, interaction with a surrounding structure, and the physical characteristics of an individual tooth, and the generating of the basic contour information may include identifying multiple predefined regions in the tooth model, calculating the clearance distance for each region corresponding to each of the multiple regions, applying the calculated clearance distance for each region to each of the multiple regions, and performing surface processing corresponding to the region where the clearance distance is applied.

The generating of the contour information may include generating mandibular advancement amount information based on oral data, and generating the contour information by adjusting a relative position between a maxilla and a mandible of the master model based on the generated mandibular advancement amount information, and the oral data may include information regarding a user's oral condition and includes impression acquisition result data and jaw movement image data.

The generating of the plurality of part models may include generating a first part model corresponding to the maxillary model based on the contour information, and generating a second part model corresponding to the mandibular model based on the contour information.

The first part model and the second part model may include at least one of a lingual space for securing an airway, a wing part formed through a shape that covers a tooth, and an open hole formed through a hole shape in one region for pressure distribution.

The generating of the oral device model may include generating an integrated model by connecting the plurality of part models into one, performing surface processing on the integrated model, and generating the oral device model by performing inner surface machining on the surface-processed integrated model based on the contour information.

The method may further include generating the oral device model by processing the tooth model as an input to a deep learning model, in which the deep learning model may be a neural network model pre-trained using a training data set composed of tooth models and oral device models of multiple users.

The method may further include generating an oral device model by outputting the generated oral device model.

According to another aspect of the present disclosure, a computing device for performing a method of manufacturing a customized oral device for sleep disorder improvement using 3D modeling is disclosed. The device includes a memory storing one or more instructions, and a processor executing the one or more instructions stored in the memory, in which the processor executes the one or more instructions to perform the method of manufacturing a customized oral device for sleep disorder improvement using 3D modeling described above.

According to another embodiment of the present disclosure, a non-transitory computer-readable recording medium storing a computer program is disclosed. The computer program may be coupled to a computer which is hardware to perform the method of manufacturing a customized oral device for sleep disorder improvement using 3D modeling.

According to another embodiment of the present disclosure, a system for manufacturing a customized oral device for sleep disorder improvement using 3D modeling is disclosed. The system includes a computing device generating an oral device model, and an output device outputting and manufacturing the oral device model, in which the computing device generates a tooth model including a maxillary model and a mandibular model, generates contour information based on the tooth model, generates a plurality of part models based on the contour information, and generates the oral device model based on the plurality of part models.

Other specific details of the present disclosure are included in the detailed description and drawings.

### BRIEF DESCRIPTION OF DRAWINGS

FIG. 1 is a schematic diagram illustrating a system for implementing a method of manufacturing a customized oral device for sleep disorder improvement using 3D modeling according to an embodiment of the present disclosure.

FIG. 2 is an exemplary diagram illustrating a state in which a user wears a customized oral device according to an embodiment of the present disclosure.

FIG. 3 is a hardware configuration diagram of a computing device performing the method of manufacturing a customized oral device for sleep disorder improvement using 3D modeling according to an embodiment of the present disclosure.

FIG. 4 is a flowchart illustrating a method of manufacturing a customized oral device for sleep disorder improvement using 3D modeling according to an embodiment of the present disclosure.

FIG. 5 is an exemplary diagram for describing a tooth model according to an embodiment of the present disclosure.

FIG. 6 is an exemplary diagram for describing a master model generated in response to the tooth model according to an embodiment of the present disclosure.

FIGS. 7 and 8 are exemplary diagrams for describing a process of setting a clearance distance for each region of a tooth according to an embodiment of the present disclosure.

FIG. 9 is an exemplary diagram for describing a process of generating contour information according to an embodiment of the present disclosure.

FIG. 10 is an exemplary diagram illustrating a wing part and a lingual space according to an embodiment of the present disclosure.

FIG. 11 is an exemplary diagram illustrating the customized oral device according to an embodiment of the present disclosure from various directions.

FIG. 12 is an exemplary diagram illustrating a process of generating an oral device model using multiple part models according to an embodiment of the present disclosure.

FIG. 13 is an exemplary diagram illustrating a process of performing correction on an integrated model according to an embodiment of the present disclosure.

### DETAILED DESCRIPTION OF EXEMPLARY EMBODIMENTS

Various embodiments will now be described with reference to the drawings. In this specification, various descriptions are presented to provide an understanding of the present disclosure. However, it will be apparent that these embodiments may be practiced without such specific descriptions

The terms “component,” “module,” “system,” and the like, as used herein, refer to computer-related entities, hardware, firmware, software, a combination of software and hardware, or an execution of software. For example, a component may be, but is not limited to, a procedure running on a processor, a processor, an object, an execution thread, a program, and/or a computer. For example, both an application running on a computing device and the computing device may be a component. One or more components may reside within the processor and/or the execution thread. One component may be localized within a single computer. One component may be distributed between two or more computers. Furthermore, these components may be executed from various computer-readable media having various data structures stored therein. Components may communicate with each other via local and/or remote processing, for example, according to signals (e.g., data from a single component that interacts with other components in a local system or a distributed system and/or data transmitted to another system over a network, such as the Internet, via signals) having one or more data packets.

In addition, the term “or” is intended to indicate an inclusive “or,” and not an exclusive “or.” That is, unless otherwise specified or clear from the context, the term “X uses A or B” is intended to mean one of its natural inclusive permutations. That is, “X uses A or B” may apply to any of the cases in which X uses A, X uses B, or X uses both A and B. Furthermore, the term “and/or” used herein should be understood to refer to and encompass all possible combinations of one or more of the associated listed items.

Furthermore, the terms “includes” and/or “including” should be understood to imply the presence of the relevant features and/or components. However, it should be understood that the terms “includes” and/or “including” do not exclude the presence or addition of one or more other features, components, and/or groups thereof. Furthermore, unless otherwise specified or clear from the context to refer to the singular form, the singular form used in the specification and claims should generally be construed to mean “one or more.”

Those skilled in the art should further appreciate that various illustrative logical blocks, configurations, modules, circuits, means, logics, and algorithm steps described in connection with the embodiments disclosed herein may be implemented as electronic hardware, computer software, or combinations of electronic hardware and computer software. To clearly illustrate the interchangeability of hardware and software, various illustrative components, blocks, configurations, means, logics, modules, circuits, and steps have been generally described above in terms of their functionality. Whether such functionality is implemented as hardware or software depends upon the particular application and design constraints imposed on the overall system. Those skilled in the art may implement the described functionality in various methods for each particular application. However, such implementation decisions should not be construed as departing from the scope of the present disclosure.

The description of the present embodiments is provided to enable those skilled in the art to which the present disclosure pertains to make or use the present disclosure. Various modifications to these embodiments will be apparent to those of ordinary skill in the art to which the present disclosure pertains. The general principles defined herein may be applied to other embodiments without departing from the scope of the present disclosure. Accordingly, the present disclosure is not limited to the embodiments set forth herein. The present disclosure should be interpreted in the broadest scope consistent with the principles and novel features set forth herein.

In this specification, “computer” means all kinds of hardware devices including at least one processor, and can be understood as including a software configuration which is operated in the corresponding hardware device according to the embodiment. For example, the meaning of “computer” may be understood to include all of smart phones, tablet PCs, desk tops, notebooks, and user clients and applications running on each device, but is not limited thereto.

Hereinafter, embodiments of the present disclosure will be described in detail with reference to the accompanying drawings.

Each step described in the present disclosure is described as being performed by a computer, but subjects of each step are not limited thereto, and according to embodiments, at least some of each step can also be performed on different devices.

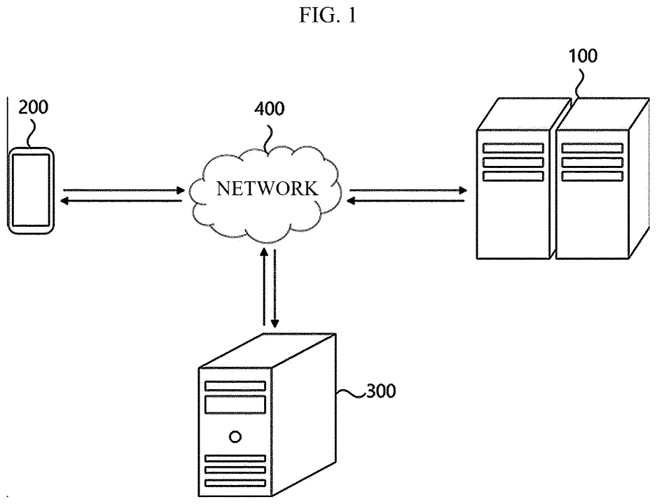

FIG. 1 is a diagram schematically illustrating a system for implementing a method of manufacturing a customized oral device for sleep disorder improvement using 3D modeling.

As illustrated in FIG. 1, a system according to embodiments of the present disclosure may include a computing device 100, a user terminal 200, an external server 300, and a network 400. The components illustrated in FIG. 1 are exemplary, and additional components may be present or some of the components illustrated in FIG. 1 may be omitted. The computing device 100, the external server 300, and the user terminal 200 according to embodiments of the present disclosure may mutually transmit and receive data for the system according to embodiments of the present disclosure via the network 400.

The network 400 according to embodiments of the present disclosure may use various wired communication systems such as a public switched telephone network (PSTN), an x digital subscriber line (xDSL), a rate adaptive DSL (RADSL), a multi rate DSL (MDSL), a very high speed DSL (VDSL), a universal asymmetric DSL (UADSL), a high bit rate DSL (HDSL), and a local area network (LAN).

In addition, the network 400 disclosed herein may use various wireless communication systems such as code division multi access (CDMA), time division multi access (TDMA), frequency division multi access (FDMA), orthogonal frequency division multi access (OFDMA), single carrier-FDMA (SC-FDMA), and other systems.

The network 400 according to embodiments of the present disclosure may be configured in any communication mode, such as wired or wireless, and may be configured as various communication networks, such as a personal area network (PAN) and a wide area network (WAN). In addition, the network 400 may be the known World Wide Web (WWW) and use a wireless transmission technology used in short range communication such as Infrared Data Association (IrDA) or Bluetooth. The technologies described herein may be used not only in the networks described above, but also in other networks.

According to an embodiment of the present disclosure, the computing device 100 (hereinafter referred to as “computing device 100”) that performs a method of manufacturing a customized oral device for sleep disorder improvement using 3D modeling may generate a customized oral device model based on an oral structure and tooth condition of an individual user.

In an embodiment, the computing device 100 may acquire oral data related to the user's oral structure and tooth condition and generate an oral device model based on the acquired oral data. For example, the computing device 100 may generate an oral device model optimized for the user's oral structure and tooth arrangement based on 3D digital data (e.g., a tooth model) generated as a result of scanning the user's impression acquisition result data. As another example, the computing device 100 may also generate a tooth model using captured data obtained by capturing an image of the user's teeth. The captured data may be acquired using an oral scanner or a camera of a user terminal (e.g., a smartphone), but is not limited thereto.

In the present disclosure, the oral device model may be a 3D model optimized for the user's oral structure and tooth condition. A customized oral device optimized for a user may be manufactured based on the oral device model, and the device may be utilized to effectively improve the user's sleep disorder. Furthermore, the oral device model may be fine-tuned based on the user's oral condition, thereby improving the device's wearing comfort and maximizing the therapeutic effectiveness related to the sleep disorder.

According to an embodiment, a device may be physically manufactured using 3D printing based on the oral device model, followed by a polishing process of smoothing a surface of the device. Thereafter, after a cleaning and inspection process is performed, the customized oral device may be finally provided to a user. This series of processes may contribute to maximizing the precision and effect of the customized oral device and effectively improving the user's sleep disorder.

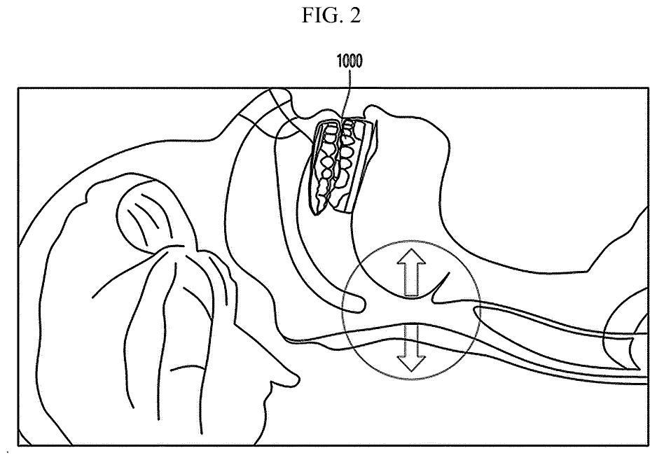

Referring to FIG. 2, the customized oral device may serve to advance a mandible to physically widen a narrowed airway. The finally generated customized oral device may be worn by a user in the mouth during sleep, thereby securing an airway to enable smooth breathing and effectively improving sleep disorders such as snoring and sleep apnea.

According to an embodiment of the present disclosure, the computing device 100 may generate individual part models corresponding to each of the maxilla and mandible, and then integrate the part models to generate an integrated oral device model. Specifically, the computing device 100 first analyzes a user's oral structure to generate the individual part models corresponding to the maxilla and mandible, and then merges these two part models to form a single, integrated oral device model. As the oral device model is generated as a one-piece structure without separate accessories for merging, the structural integrity may be increased to minimize the risk of breakage or deformation during use and provide easy maintenance.

In an embodiment, the computing device 100 of the present disclosure may adjust the arrangement distance between the maxilla and mandible based on the user's oral data, and precisely adjust positions of individual part models accordingly, thereby generating the optimized oral device model.

The oral data may include various data related to the user's oral condition, and may include, for example, data on the user's impression acquisition results, a plurality of pieces of oral image data obtained by capturing images of the oral structure from multiple angles, and jaw movement image data. The data may play a key role in designing the customized oral device model for each user, thereby providing a device optimized for each user's oral structure.

That is, the computing device 100 may comprehensively analyze the user's oral data to design and generate an integrated oral device model through precise arrangement of the maxilla and mandible and optimized positional adjustment between the part models. This may improve the wearing comfort and durability of the customized oral device and enhance its therapeutic effectiveness in improving sleep disorders.

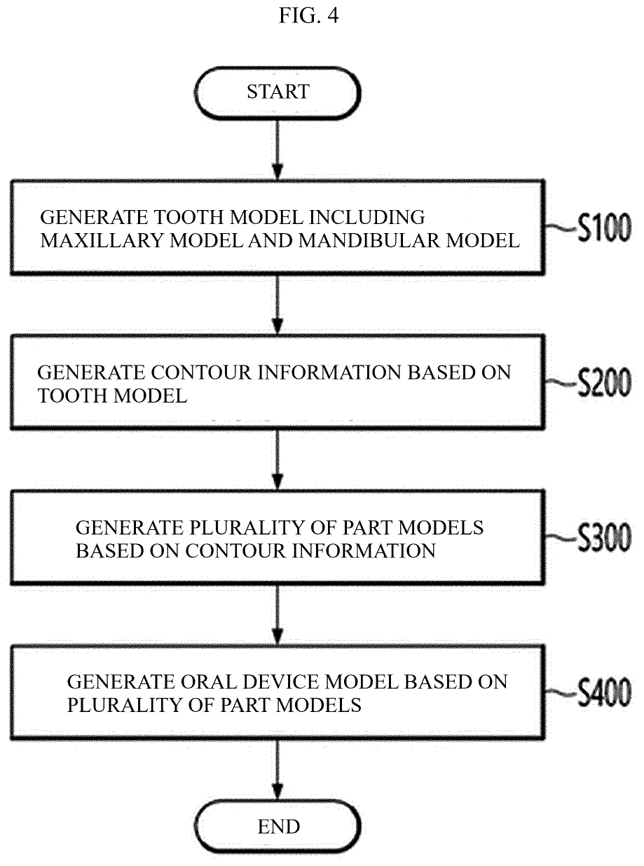

Furthermore, according to an embodiment of the present disclosure, by applying the clearance distance related to each user's oral structure and tooth arrangement during the oral device model generation process, the pressure during wearing may be evenly distributed without being concentrated on a specific area. Specifically, this clearance design may be customized and applied to each user, taking into account their unique oral structures and tooth arrangements, thereby enabling the pressure applied to each tooth to be optimized. This may reduce fatigue and discomfort even when the device is worn for long periods of time, and maintain a natural and stable wearing state even during sleep. As a result, long-term therapeutic effects may be maintained while the device is being used, and a higher effect in improving sleep disorders may be expected. A detailed description of the method of manufacturing a customized oral device for sleep disorder improvement using 3D modeling related to the present disclosure will be described below with reference to FIGS. 4 to 13.

In an embodiment, although only one computing device 100 is illustrated in FIG. 1, it will be apparent to those skilled in the art that more servers may also be included within the scope of the present disclosure, and that the computing device 100 may include additional components. That is, the computing device 100 may be composed of a plurality of computing devices. In other words, a set of multiple nodes may constitute the computing device 100.

According to an embodiment of the present disclosure, the computing device 100 may be a server providing a cloud computing service. More specifically, the computing device 100 may be a server that provides a cloud computing service, which is a type of Internet-based computing, for processing information regarding other computers connected to the Internet rather than the user's computer. The cloud computing service may be a service that stores data on the Internet and allows users to access and use necessary data or programs anytime and anywhere via an Internet connection without installing the data or programs on their computers. The cloud computing service may allow users to easily share and transmit data stored on the Internet with simple operations and clicks. Furthermore, the cloud computing service may be a service that may not only store data in servers on the Internet, but also allow users to perform desired tasks using functions of application programs provided from a web without installing separate programs, and allow multiple people to simultaneously share and work on documents. In addition, the cloud computing service may be implemented in at least one of infrastructure as a service (IaaS), platform as a service (PaaS), software as a service (SaaS), a virtual machine-based cloud server, and a container-based cloud server. That is, the computing device 100 of the present disclosure may be implemented in at least one of the above-described cloud computing services. The specific description of the above-described cloud computing service is merely exemplary, and any platform that constructs the cloud computing environment of the present disclosure may be included.

According to an embodiment of the present disclosure, the user terminal 200 may be a terminal associated with a user who accesses the computing device 100 to acquire analysis information regarding 3D modeling data (e.g., data on the individual user's oral structure), such as a customized oral device model or an optimized device design plan. For example, the user terminal 200 may be a terminal associated with a user (e.g., a dentist) who provides the design results of the customized oral device to the user (e.g., a patient). When the user terminal 200 is a terminal associated with a dentist who provides customized oral device design results to a patient, the 3D modeling analysis information provided from the computing device 100 may be utilized as a medical auxiliary terminal for diagnosis and device design establishment. The user terminal 200 is equipped with a display, and thus may receive user input and provide output of any form to a user.

The user terminal 200 may transmit the stored oral data of the patient to the computing device 100, and the computing device 100 may generate analysis results or a customized oral device design based on the data and may provide the analysis results or customized oral device design to the user terminal 200.

In an embodiment, the user terminal 200 may be any form of entity(ies) in a system having a mechanism for communicating with the computing device 100. For example, the user terminal 200 may include a personal computer (PC), a notebook, a mobile terminal, a smart phone, a tablet PC, a wearable device, etc., and may include all types of terminals capable of connecting to wired/wireless networks. In addition, the user terminal 200 may include any server implemented by at least one of an agent, an application programming interface (API), and a plug-in. In addition, the user terminal 200 may include application sources and/or client applications.

In an embodiment, the external server 300 may be connected to the computing device 100 via the network 400, and may provide various pieces of information and data necessary for the computing device 100 to perform a 3D modeling method of a user-customized oral device or receive, store, and manage optimized oral device design data derived from the 3D modeling process. For example, the external server 300 may be a storage server separately provided outside the computing device 100, but is not limited thereto.

According to an embodiment of the present disclosure, the external server 300 may be a server that stores oral scan data, tooth arrangement data, and treatment plan data related thereto that correspond to multiple users. Specifically, the external server 300 may store and manage 3D scan data of the user's oral structure, orthodontic modeling data, and dental diagnostic findings or customized oral device design data related thereto. As another example, the external server 300 may store a manufacturing history of each user's oral device and device usage history data, and may link and store treatment results based on this. This information may be utilized to comprehensively review the user's entire treatment process and continuously improve the design of the optimized customized oral device.

For example, the external server 300 may be at least one of a dental hospital server and an oral device manufacturer's server, and may be a server that stores information regarding the user's oral diagnosis data and customized oral device design. Furthermore, the external server 300 may be a server of an oral device-related research institute or a medical data management institution, and serve to manage and store research results and device-effect analysis data related to the user's oral data, which are generated by such institutions.

The information stored in the external server 300 may be utilized as training data, verification data, and test data for training the AI-based 3D modeling system of the present disclosure. That is, the external server 300 may store a data set for training a neural network model of the present disclosure, thereby improving the accuracy of oral device modeling and design. The computing device 100 of the present disclosure may acquire various oral data (or tooth data) from the external server 300 and construct a training data set based on the oral data, thereby developing an artificial intelligence model capable of generating a more precise customized oral device model.

The information stored in the external server 300 may be utilized as the training data, verification data, and test data for training the neural network of the present disclosure. That is, the external server 300 may be a server that stores information regarding a data set for training the neural network model of the present disclosure. The external server 300 may store data for training the artificial intelligence model of the present disclosure. The computing device 100 of the present disclosure may acquire multiple medical data sets from the external server 300 and construct multiple training data sets based on the acquired medical data. The computing device 100 may perform training using the data included in the training data set, thereby generating an artificial intelligence model.

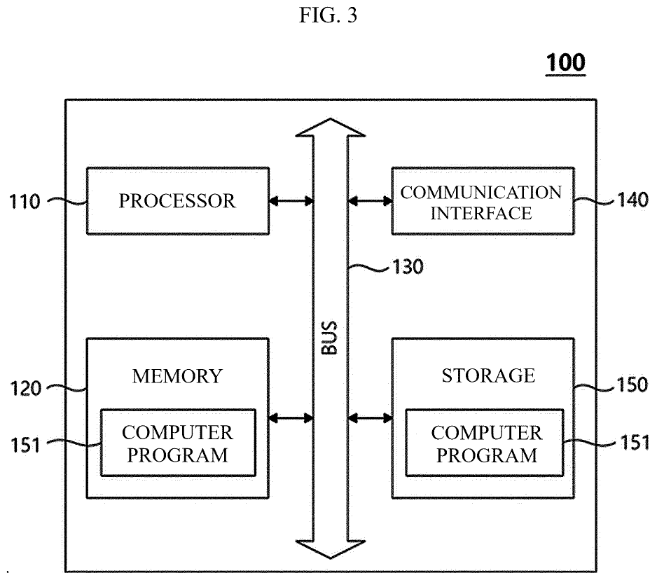

The external server 300 is a digital device, and may be a digital device equipped with a processor, and a memory, such as a laptop computer, notebook computer, desktop computer, web pad, or mobile phone. The external server 300 may be a web server that processes services. The above-described types of servers are merely examples, and the present disclosure is not limited thereto. Hereinafter, the hardware configuration of the computing device 100 that performs the method of manufacturing a customized oral device for sleep disorder improvement using 3D modeling will be described with reference to FIG. 3.

FIG. 3 is a hardware configuration diagram of the computing device that performs the method of manufacturing a customized oral device for sleep disorder improvement using 3D modeling according to an embodiment of the present disclosure.

Referring to FIG. 3, the computing device 100 that performs the method of manufacturing a customized oral device for sleep disorder improvement using 3D modeling according to an embodiment of the present disclosure may include one or more processors 110, a memory 120 that loads a computer program 151 executed by the processor 110, a bus 130, a communication interface 140, and a storage 150 that stores the computer program 151. Here, only the components related to the embodiment of the present invention are illustrated in FIG. 2. Accordingly, those skilled in the art to which the present invention pertains may understand that general-purpose components other than those illustrated in FIG. 3 may be further included.

According to an embodiment of the present disclosure, the processor 110 typically processes the overall operation of the computing device 100. The processor 110 may provide or process appropriate information or a function to a user or a user terminal by processing signals, data, information, and the like, which are input or output through the above-described components, or by driving an application program stored in the memory 120.

In addition, the processor 110 may perform an operation on at least one application or program for executing the method according to the embodiments of the present invention, and the computing device 100 may include one or more processors.

According to an embodiment of the present disclosure, the processor 110 may be configured with one or more cores, and may include a processor for data analysis and deep learning, such as a central processing unit (CPU), a general purpose graphics processing unit (GPGPU), or a tensor processing unit (TPU) of a computing device.

The processor 110 may read a computer program stored in the memory 120 and perform data processing for the artificial intelligence model according to an embodiment of the present disclosure. According to an embodiment of the present disclosure, the processor 110 may perform an operation for training a neural network. The processor 110 may perform calculations for training a neural network, such as processing input data for training in deep learning (DL), extracting features from input data, calculating errors, and updating weights of a neural network using backpropagation.

In addition, at least one of the CPU, GPGPU, and TPU of the processor 110 may process training of a network function. For example, the CPU and the GPGPU may jointly process training of network function and data classification using the network function. Furthermore, in an embodiment of the present disclosure, processors of multiple computing devices may be used together to process the training of the network function and the data classification using the network function. In addition, a computer program executed on a computing device according to an embodiment of the present disclosure may be a CPU, GPGPU, or TPU executable program.

In this specification, the network function may be used interchangeably with an artificial neural network or a neural network. In this specification, the network function may include one or more neural networks. In this case, the output of the network function may be an ensemble of outputs of one or more neural networks.

The processor 110 may read a computer program stored in the memory 120 and provide a deep learning model according to an embodiment of the present disclosure. According to an embodiment of the present disclosure, the processor 110 may perform a calculation for training a deep learning model.

According to an embodiment of the present disclosure, the processor 110 typically processes the overall operation of the computing device 100. The processor 110 may provide or process appropriate information or a function for a user or a user terminal by processing signals, data, information, and the like, which are input or output through the above-described components, or by driving an application program stored in the memory 120.

In addition, the processor 110 may perform an operation on at least one application or program for executing the method according to the embodiments of the present invention, and the computing device 100 may include one or more processors.

According to various embodiments, the processor 110 may further include a random access memory (RAM) (not illustrated) and a read-only memory (ROM) for temporarily and/or permanently storing signals (or data) processed in the processor 110. In addition, the processor 110 may be implemented in the form of a system-on-chip (SoC) including at least one of a graphics processing unit, a RAM, and a ROM.

The memory 120 stores various types of data, commands and/or information. The memory 120 may load the computer program 151 from the storage 150 to execute methods/operations according to various embodiments of the present invention. When the computer program 151 is loaded into the memory 120, the processor 110 may perform the method/operation by executing one or more instructions constituting the computer program 151. The memory 120 may be implemented as a volatile memory such as a RAM, but the technical scope of the present disclosure is not limited thereto.

The bus 130 provides a communication function between the components of the computing device 100. The bus 130 may be implemented as various types of buses, such as an address bus, a data bus, and a control bus.

The communication interface 140 supports wired/wireless Internet communication of the computing device 100. In addition, the communication interface 140 may support various communication methods other than the Internet communication. To this end, the communication interface 140 may be configured to include a communication module well known in the art of the present invention. In some embodiments, the communication interface 140 may be omitted.

The storage 150 may non-temporarily store the computer program 151. When performing a process for 3D modeling of a customized oral device for sleep disorder improvement through the computing device 100, the storage 150 may store various pieces of information necessary to provide the process for 3D modeling of a customized oral device for sleep disorder improvement.

The storage 150 may include a nonvolatile memory, such as a ROM, an erasable programmable ROM (EPROM), an electrically erasable programmable ROM (EEPROM), and a flash memory, a hard disk, a removable disk, or any well-known computer-readable recording medium in the art to which the present invention pertains.

The computer program 151 may include one or more instructions to cause the processor 110 to perform methods/operations according to various embodiments of the present invention when loaded into the memory 120. That is, the processor 110 may perform the method/operation according to various embodiments of the present invention by executing the one or more instructions.

In an embodiment, the computer program 151 may include one or more instructions to perform a method of manufacturing a customized oral device for sleep disorder improvement using 3D modeling, including: generating a tooth model including a maxillary model and a mandibular model; generating contour information based on the tooth model; generating a plurality of part models based on the contour information; and generating an oral device model based on the plurality of part models.

Operations of the method or algorithm described with reference to the embodiment of the present disclosure may be directly implemented in hardware, in software modules executed by hardware, or in a combination thereof. The software module may reside in a random access memory (RAM), a read only memory (ROM), an erasable programmable ROM (EPROM), an electrically erasable programmable ROM (EEPROM), a flash memory, a hard disk, a removable disk, a compact disc read-only memory (CD-ROM), or in any form of computer-readable recording medium known in the art to which the invention pertains.

The components of the present disclosure may be embodied as a program (or application) and stored in a medium for execution in combination with a computer which is hardware. The components of the present disclosure may be executed in software programming or software elements, and similarly, embodiments may be realized in a programming or scripting language such as C, C++, Java, and assembly, including various algorithms implemented in a combination of data structures, processes, routines, or other programming constructions. Functional aspects may be implemented in algorithms executed on one or more processors. The method of manufacturing a customized oral device for sleep disorder improvement using 3D modeling performed by a computing device 100 will be described in detail below with reference to FIGS. 4 to 13.

FIG. 4 illustrates a flowchart exemplarily illustrating the method of manufacturing a customized oral device for sleep disorder improvement using 3D modeling according to an embodiment of the present disclosure. The order of the operations illustrated in FIG. 4 may be changed as necessary, and at least one operation may be omitted or added. In other words, the following operations are merely exemplary embodiments of the present disclosure, and the scope of the present disclosure is not limited thereto.

According to an embodiment of the present disclosure, the method of manufacturing a customized oral device for sleep disorder improvement using 3D modeling may include acquiring oral data for generating a tooth model.

In an embodiment, the oral data may include various data related to the user's oral structure and tooth condition. For example, the oral data may include, but is not limited to, the user' impression acquisition result data, 3D scan data, tooth arrangement information, and jaw movement image data.

Specifically, the impression acquisition result data is a digital conversion of physical impression data that accurately replicates the user's oral structure, and accurately reflects the shape, size, arrangement, etc., of teeth to provide the basis for 3D modeling.

The 3D scan data is a three-dimensional digital image generated by scanning the user's oral structure from various angles, and may provide more precise structural information than impression acquisition data and may even include detailed shapes of teeth and gums. This allows for accurate modeling of the relationship between teeth and surrounding tissues.

The tooth arrangement data, which represents the position and alignment of each tooth, plays a key role in analyzing occlusion and interactions between teeth, and may also help ensure that the oral device is designed to fit the arrangement of the teeth and perform its functions effectively.

The jaw movement image data is video recordings that capture the movements of a user's jaw, and may be utilized to analyze the dynamic relationship between the mandible and the maxilla. The jaw movement image data helps ensure natural operation of the device when worn by considering the jaw's movement during the oral device design, and also plays a key role in improving device stability and wearing comfort by accurately reflecting the user's bite force and range of jaw motion.

This oral data provides essential information for the computing device 100 to precisely design the user-customized oral device model and contributes to the generation of devices optimized for sleep disorder improvement.

In an embodiment, acquiring the oral data may include receiving or loading the data stored in the memory 120. Acquiring the oral data may include receiving or loading the oral data in or into another storage medium, or a separate processing module within the same computing device or another computing device, based on wired/wireless communication means. For example, a user may access the computing device 100 via the user terminal 200, is provided with a user interface related to acquiring the oral data from the computing device 100, and may transmit the oral data to the computing device 100 by dragging and dropping the oral data onto the provided user interface. As another example, the computing device 100 may be linked to the external server 300 to automatically retrieve the oral data stored in the external server 300, or the user may transmit specific oral data to the external server 300 for storage. This may allow for more efficient and automated acquisition of the oral data, and allow a user to conveniently manage and utilize the data.

According to an embodiment of the present disclosure, the method of manufacturing a customized oral device for sleep disorder improvement using 3D modeling may include generating a tooth model including a maxillary model and a mandibular model (S300).

In an embodiment, the tooth model is a 3D model that precisely reflects the user's oral structure and tooth arrangement, and may accurately represent the entire oral structure, including individual models corresponding to each of the maxilla and mandible.

In an embodiment, the computing device 100 may generate the tooth model based on the impression acquisition result data. The impression acquisition result data is a digital conversion of the physical impression data that accurately replicates the user's oral structure, and may accurately reflect the shape, size, position, etc., of teeth based on the corresponding data. For example, a plaster stone is manufactured by pouring plaster into the impression acquisition result corresponding to the user, and then digitized and converted into a 3D model, thereby precisely reproducing the user's oral structure. In an embodiment, high-resolution digital data, i.e., the impression acquisition result data, may be acquired from the impression acquisition result through a scanning or image processing process. During this process, the physical impression acquisition results are converted into a highly precise 3D shape, thereby generating data that includes the fine structure and surface details of teeth. This generates the digitized 3D model, which may then be used as the basis for designing the customized oral device model.

Furthermore, in an embodiment, the computing device 100 may generate a tooth model based on multiple oral images captured from various angles. For example, a user utilizes a user terminal to perform a procedure of capturing images of his/her oral conditions from various angles through a user interface provided from the computing device 100, thereby acquiring multiple oral images. These oral images are used as data for precisely understanding the tooth arrangement and the relationships between the maxilla and the mandible.

According to an embodiment, when necessary data is insufficient during the oral image acquisition process or images of a specific area are insufficient, the computing device 100 may guide a user through an operation of acquiring additional images. In this way, by supplementing the required additional oral images, a more accurate and complete tooth model may be generated.

More specifically, the computing device 100 may analyze data of multiple acquired oral images to assess the quality and completeness of the images. The computing device 100 may check whether each image is clearly captured, whether all teeth and oral structures are clearly identifiable, and whether critical information regarding specific oral areas or tooth arrangements is missing. When an image of a specific area is unclear or a portion of a major oral structure is not captured, the computing device 100 may automatically identify the corresponding area and determine whether additional image capturing is necessary.

Furthermore, the computing device 100 provides a user interface that includes information clearly explaining the areas requiring additional capturing and the reasons for such acquisition. The user interface may provide specific capturing guidelines to a user, and indicate, for example, a capturing angle, a specific location within an oral cavity, etc. The user may additionally capture images of the missing area based on the guidance provided through the user interface and transmit the captured images to the computing device 100.

Through this process, the computing device 100 may completely capture all necessary oral images, thereby generating a more accurate and precise tooth model based on the captured oral images. This precise tooth model serves as the basis for designing the oral device and plays a key role in providing the customized oral device optimized for the user.

The computing device 100 may accurately reproduce the fine structure and arrangement of teeth by acquiring multiple oral images and converting the oral images into a 3D model based on the acquired oral images.

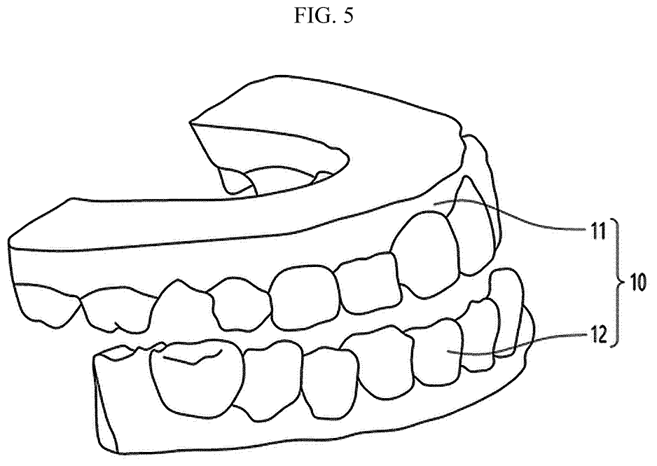

In an embodiment, as illustrated in FIG. 5, a tooth model 10 may include a maxillary model 11 and a mandibular model 12. FIG. 5 is an exemplary diagram for describing a tooth model according to an embodiment of the present disclosure.

The tooth model 10 precisely reflects the user's oral structure, thereby accurately representing the occlusal relationship between the maxilla and the mandible. In addition, the tooth model 10 may reproduce the position and size of each tooth, and a relationship of each tooth with the surrounding periodontal tissue.

In various embodiments, the computing device 100 may generate the tooth model 10 in which the positional relationship between the maxilla and the mandible is adjusted to reflect the mandibular advancement amount for the user, but is not limited thereto.

According to an embodiment of the present disclosure, the method of manufacturing a customized oral device for sleep disorder improvement using 3D modeling may include generating the contour information based on the tooth model (S200).

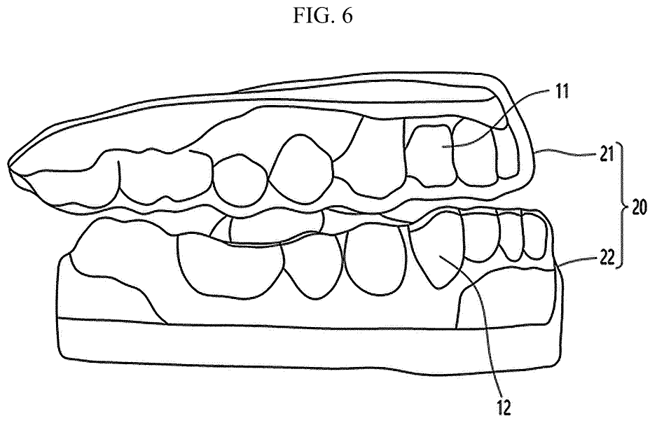

In an embodiment, the generating of the contour information may include generating a master model by applying a clearance distance corresponding to each of multiple regions of the tooth model 10, generating basic contour information based on the master model, and generating the contour information by correcting the master model based on the basic contour information. According to an embodiment, a master model 20 is generated in response to the tooth model 10, and as illustrated in FIG. 6, may include a first master sub-model 21 corresponding to the maxillary model 11 and a second master sub-model 22 corresponding to the mandibular model 12.

In an embodiment, the clearance distance may be set to allow for pressure distribution and a predetermined amount of movement, and determined differently for each region based on anatomical characteristics of a tooth, interaction with a surrounding structure, and the physical characteristics of an individual tooth.

In an embodiment, the basic contour information is generated based on the contour of the master model generated by applying the clearance distance to the tooth model corresponding to the user.

The contour information is generated by adjusting the arrangement distance between the maxilla (i.e., the first master sub-model) and the mandible (i.e., the second master sub-model) of the master model based on the basic contour information. In particular, the contour information is utilized to design an oral device that focuses on advancing the mandible to secure an airway, thereby enabling smooth breathing during sleep.

In an embodiment, the contour information (or basic contour information) does not directly represent the physical thickness or volume of the oral device, but rather may be composed of linear data defined along the perimeter of the master model. In other words, the contour information does not represent structural elements of the actual oral device, but is a virtual contour used to define the shape of the device, and may serve as a basic reference line for the optimal arrangement of the maxilla and mandible during the design stage.

More specifically, the computing device 100 analyzes the characteristics of each tooth and the surrounding tissue to identify the areas where the clearance distance is required or appropriate. This prevents excessive pressure from being concentrated on specific teeth, thereby minimizing discomfort when the device is worn and allowing natural movement of teeth and gums. Based on these analysis results, the computing device 100 may generate the master model, and optimize the positions of the maxilla and mandible to generate the contour information.

In a specific embodiment, the generating of the basic contour information may include identifying multiple predefined regions in a tooth model, calculating a clearance distance for each region corresponding to each of the multiple regions, applying the calculated clearance distance for each region to each of the multiple regions, and performing surface processing corresponding to the region where the clearance distance has been applied.

The computing device 100 may first form a clearance (e.g., a basic clearance) in the basic tooth and gum structure to facilitate the detachment of the oral device, and then identify predefined regions and provide appropriate clearance to each identified region.

In an embodiment, the predefined regions may be set as multiple regions for each of the maxilla and mandible, and may be regions defined to maximize comfort and functionality when the oral device is worn.

In an embodiment, for the mandible, the predefined regions may include a first region corresponding to a mandibular anterior teeth abutment surface and a second region corresponding to a mandibular molar abutment surface. The first region is an area where pain is likely to occur when the device is worn, and a relatively large clearance distance is assigned to distribute pressure. The second region is the molar abutment surface, and is assigned the clearance distance to allow a small range of movement.

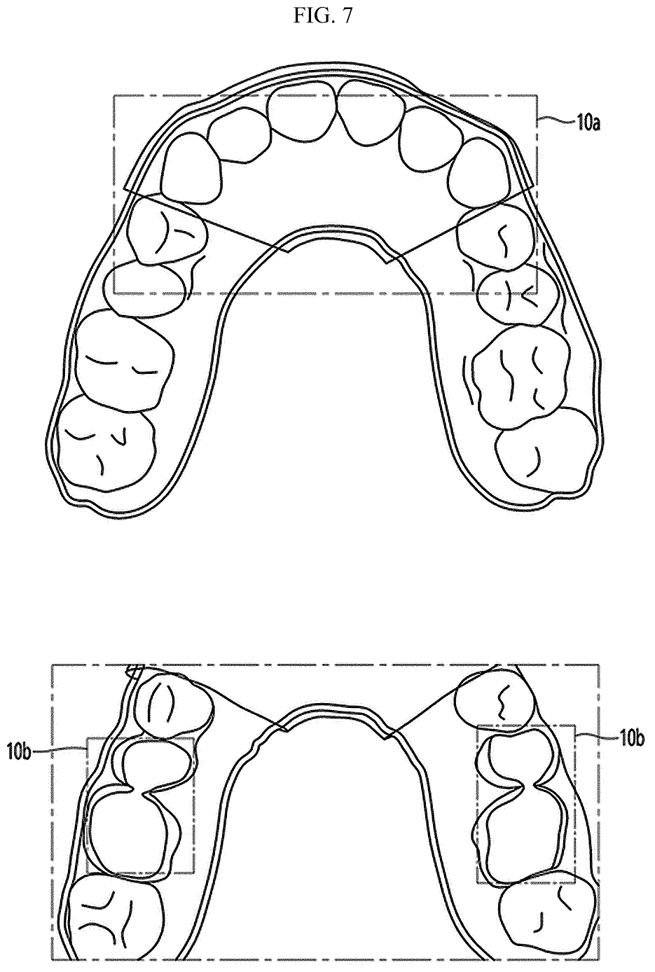

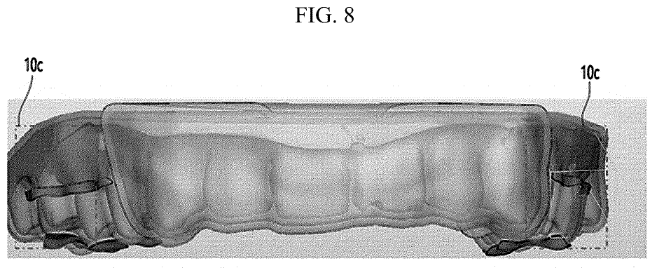

In an embodiment, for the maxilla, the predefined regions may include a third region corresponding to the maxillary anterior teeth, a fourth region corresponding to the maxillary molar abutment surface, a fifth region corresponding to an outer surface of the maxilla, and a sixth region corresponding to an outer surface of the anterior teeth. As a specific example, referring to FIG. 7, the third region 10a associated with the maxillary anterior teeth is an area where pain is most likely to occur, and is assigned the clearance distance to minimize discomfort when the device is worn. The fourth region 10b associated with the maxillary molar abutment surface is an area where the oral device is likely to be damaged due to strong bite force being applied, and is assigned the clearance distance to prevent damage. Referring to FIG. 8, the fifth and sixth regions 10c associated with the outer surface of the maxilla and the outer surface of the anterior teeth are portions where the oral device may catch during detachment, and is assigned an appropriate clearance distance to facilitate the detachment.

According to the embodiment, the clearance distances are set to different sizes depending on the predefined regions, maximizing comfort and functionality when the oral device is worn.

As a specific example, the area set as the first region in the mandible is an area that is bonded to the mandibular anterior teeth and is highly susceptible to pain when the device is worn. This portion is assigned the relatively largest clearance distance, Clearance 1, to prevent pressure from being concentrated in this area and minimize discomfort during wear. The second region of the mandible corresponds to the mandibular molar abutment. This area requires a small range of movement, and is set with the second clearance distance which is a medium clearance distance. Since tooth-to-tooth interaction is crucial in this area, appropriate clearance is assigned to allow movement while maintaining the stability of the device.

In the case of the maxilla, the maxillary anterior teeth set as the third region are an area where the pain is highly likely to occur and are assigned the first clearance distance that is the largest clearance distance (the first clearance distance) compared to all other areas. This ensures even pressure distribution, improving wearing comfort and minimizing discomfort during long-term wear. The maxillary molar abutment, which is the fourth region, is an area where the strongest bite force is applied, and is a region where the oral device is highly likely to be damaged. The second clearance distance, which is the medium clearance distance, is assigned to this area to prevent damage and enhance device durability.

Furthermore, the outer side surface (the fifth region) of the maxilla and the outer surface (the sixth region) of the anterior teeth are portions where the oral device may be caught during detachment. These areas may be assigned a third clearance distance which is a relatively small clearance distance. This small clearance distance reduces friction that may occur during wearing and removal of the device, facilitating easy detachment.

In this way, by relatively comparing and setting the clearance distances for each region, the computing device 100 may design the oral device in the form optimized for the user. By assigning greater clearance to areas requiring greater clearance, medium clearance to areas requiring stability and durability, and small clearance to areas requiring easy detachment, it is possible to provide a stable wearing experience with minimal discomfort even during long-term wear. These clearance settings also enhance the durability of the device, minimize potential damage during use, and maximize therapeutic effectiveness by ensuring that the device adheres properly to the teeth and gums.

In various embodiments, the computing device 100 may acquire various oral data from the user terminal, and apply additional corrections to the clearance distance corresponding to each area based on the acquired oral data. This additional correction reflects the user's oral characteristics and individual requirements to provide optimal wearing comfort.

First, the computing device 100 collects various oral data, including an impression acquisition result, tooth sensitivity, occlusal force, a gum condition, and jaw movement image information of the user. The impression acquisition result provides information that enables precise identification of the user's tooth structure and tooth arrangement, and tooth sensitivity and gingival condition may be acquired through survey data, clinical records, etc. The occlusal force and the jaw movement image data are collected by analyzing the force and patterns exerted when the user bites or moves their teeth.

Based on this data, the computing device 100 initially sets the clearance distance for each area and then analyzes the collected data to determine the degree of correction. Specifically, the computing device 100 may generate tooth sensitivity information, gum condition information, occlusal force information, and tooth structure information based on the acquired oral data, and may analyze correlations within the information to calculate an optimal correction value.

In an embodiment, the computing device 100 may generate tooth sensitivity information and gum condition information based on clinical records and questionnaire data obtained from the user. The tooth sensitivity information reflects the areas where the user perceives sensitivity, indicating the degree to which specific teeth or gums are sensitive to pressure. Gum condition information includes factors such as gum thickness, health status, and presence of inflammation, and incorporates data to predict how the gums will respond to pressure or device wear.

Furthermore, the computing device 100 may generate occlusal force information by analyzing the user's jaw movement image data and occlusal condition. The occlusal force information is data obtained by measuring the magnitude and direction of the force generated when the user bites with their teeth, and evaluating the contact area between the maxilla and the mandible, the balance of the biting force, interactions between teeth, etc., to be reflected in the oral device design.

Furthermore, the computing device 100 may generate tooth structure information using 3D scan data. This tooth structure information is information that represents not only the shape, size, and arrangement of each tooth, but also the surface area of the tooth and the relationship with surrounding tissues. Through this data, the oral device is designed to accurately fit to each tooth.

According to an embodiment, the first clearance distance corresponding to the bonding surface of the mandibular anterior teeth and the maxillary anterior teeth may be corrected based on the analysis of the user's tooth sensitivity information and gum condition information. For users with high tooth sensitivity, the computing device 100 may apply additional corrections such that the determined clearance distance is increased further to ensure that the pressure applied to the gums does not cause discomfort.

Additionally, in the embodiment, the second clearance distance corresponding to the maxillary molar interface may be corrected based on the user's occlusal force and tooth structure information. For users with strong occlusal force, the computing device 100 may apply additional corrections to widen the clearance distance in order to alleviate the impact between the teeth and the oral device. This reduces wear and damage that may occur when the device is worn and contributes to maintaining stable interaction between the user's teeth and the device. On the other hand, for users with weak occlusal force, the clearance distance may be reduced to increase the seal between the teeth and the device, thereby improving wearing comfort and enhancing device stability.

Furthermore, the third clearance distance corresponding to the outer side surface of the maxilla and the outer surface of the anterior teeth may be corrected to facilitate device wearing and detachment. The computing device 100 analyzes the jaw movement image information and adjusts the clearance distance to reduce friction that may occur when the oral device is attached and detached. In the area where excessive friction occurs, a smaller clearance distance is applied to alleviate friction, and the device can be easily detached.

In this way, the computing device 100 may analyze various oral data and perform additional corrections to the clearance distance determined for each area to generate basic contour information. The basic contour information is the result of reflecting the user's tooth and gum structure, sensitivity, occlusal force, jaw movement, etc., and applying additional corrections to the clearance tailored to each region, and allows the oral device to be designed to be optimized for the user.

Furthermore, in an embodiment, the computing device 100 may perform surface processing corresponding to the region where the clearance distance is applied. The surface processing of the clearance portion plays a key role in maximizing the wearing comfort of the oral device and preventing pressure from being concentrated in a specific area. During this process, the computing device 100 may smooth the surface of the portion where the clearance is applied or remove unnecessary protrusions, thereby ensuring that the device smoothly adheres to the teeth and gums.

For example, the region where the clearance is applied requires smoother contact with the teeth to distribute pressure. To this end, the computing device 100 applies surface processing techniques, such as wrapping, to remove roughness or sharp edges from the device surface, thereby ensuring that the device may be conveniently worn within the user's oral cavity. In particular, the clearance area should be designed to evenly distribute pressure the device is worn, and thus it is possible to enhance the device's finish quality through the surface processing and minimize discomfort during long-term wear.

This surface processing process ensures that the device adheres properly to the teeth and gums, reducing friction and irritation and contributing to minimizing discomfort experienced by the user. Consequently, the surface processing may improve the overall wearing comfort of the oral device, increase its durability, and contribute to protecting the user's oral health.

Furthermore, in an embodiment, the generating of the contour information may include generating mandibular advancement amount information based on oral data, and adjusting the relative position between the maxilla and the mandible of the master model based on the generated mandibular advancement amount information to generate the contour information. In an embodiment, the oral data may include information regarding a user's oral condition and include the impression acquisition result data and the jaw movement image data.

In an embodiment, the computer device 100 may analyze the impression acquisition result data and accurately assesses the user's oral structure and tooth alignment. During this process, the computing device 100 comprehensively analyzes the size, shape, and arrangement of each tooth, as well as the height and thickness of the gums and the relationship between the teeth and gums, thereby precisely reproducing the user's oral structure as a 3D model.

Additionally, the jaw movement image data provides critical data for analyzing how the user's mandible moves. Using these images, the computing device 100 precisely analyzes the range and pattern of forward, backward, up, down, left, and right movements of the mandible. For example, when the user opens and closes their mouth, the computing device 100 may determine how far the mandible advances, the angle at which the mandible moves, and how this movement interacts with the maxilla. These analysis results are used to accurately calculate the mandibular advancement amount, which is a critical factor to optimize the securing of the user's airway.

The calculated mandibular advancement amount information plays a key role in adjusting the relative position between the maxilla (i.e., the first master sub-model) and the mandible (i.e., the second master sub-model) of the master model. Based on the mandibular advancement amount information, the computing device 100 adjusts the relative position of the maxilla and mandible to ensure optimal occlusion while ensuring a smooth airway. This allows the oral device to be designed to sufficiently open the user's airway during sleep.

In an embodiment, the computing device 100 may generate the contour information by reflecting the mandibular advancement amount information in the basic contour information. In other words, by reflecting the mandibular advancement amount information to determine the optimal arrangement between the maxilla and the mandible of the master model, the oral device may assist in moving the mandible forward to secure an airway and provide an appropriate space.

Specifically, the computing device 100 analyzes the jaw movement image data to calculate the mandibular advancement amount. During this process, the computing device 100 precisely tracks the movement pattern of the mandible when the user opens and closes their mouth. The computing device 100 analyzes how the mandible moves forward, backward, up, down, left, and right when the user moves the mandible, and in particular, measures the forward movement distance of the mandible, which is important for securing the airway.

First, the computing device 100 calculates the maximum forward distance the mandible may move and measures the angle at which the mandible is most forward. To analyze how the user's mandible affects the airway at this position, the computing device evaluates the size and degree of change in the airway space. For example, the computing device analyzes how the airway expands when the mandible advances 2 mm, as well as the impact of this expansion on the occlusion of the maxilla and mandible.

Furthermore, the computing device 100 simulates how the effectiveness of securing the airway changes when the mandible advances excessively or insufficiently. By comparing the cases where the mandible advances mm and 3 mm, in each situation, the computing device 100 calculates the degree of airway patency, the user's occlusal condition, and the stress applied to the temporomandibular joint. In this way, the computing device 100 calculates the optimal amount of mandibular advancement, which is adjusted to secure the airway suitable for the user while minimizing discomfort during long-term wear.

For example, the computing device 100 may confirm as the result of analyzing the user's jaw movement image that when the mandible advances an average of 2.5 mm, the airway is maximized and the occlusion between the maxilla and the mandible remains stable. On the other hand, the analysis also finds that when the mandible advances more than 3 mm, an excessive load may be imposed on the temporomandibular joint. Based on this, the computing device 100 sets the mandibular advancement amount to 2.5 mm and reflects the set mandibular advancement amount in the design of the oral device to maximize the securing of the user's airway and maintain the occlusion stability.

In this way, the computing device 100 accurately calculates the mandibular advancement amount based on the jaw movement image, thereby securing the optimal airway access and wearing comfort.

Furthermore, in the embodiment, the computing device 100 is designed to prevent excessive mandibular advancement, thereby preventing strain on the temporomandibular joint. For example, when the mandibular advancement amount is excessive, unnecessary stress may be imposed on the temporomandibular joint. Therefore, the computing device 100 may calculate the optimal advancement amount based on the analysis results of the jaw movement image data, considering the user's temporomandibular joint condition and muscle strength, and generate the contour information based on the optimal advancement amount.

The computing device 100 analyzes the user's temporomandibular joint data and corrects the mandibular advancement amount so that the mandibular advancement amount does not exceed an appropriate range. In this case, the computing device 100 applies a design that minimizes unnecessary stress caused by the mandibular advancement by considering the muscle tension and joint movement patterns that occur when the user moves the jaw. For example, when the user experiences that advancement exceeding 2.5 mm may strain the temporomandibular joint, the computing device 100 takes this into account and sets an optimal value that limits the mandibular advancement to 2.0 to 2.5 mm. Through this, the oral device is designed to maintain an appropriate advancement amount for securing an airway while preventing the unnecessary strain on the temporomandibular joint even during long-term wear. The specific numerical description of the above-described mandibular advancement amount is merely an example, and the present disclosure is not limited thereto.

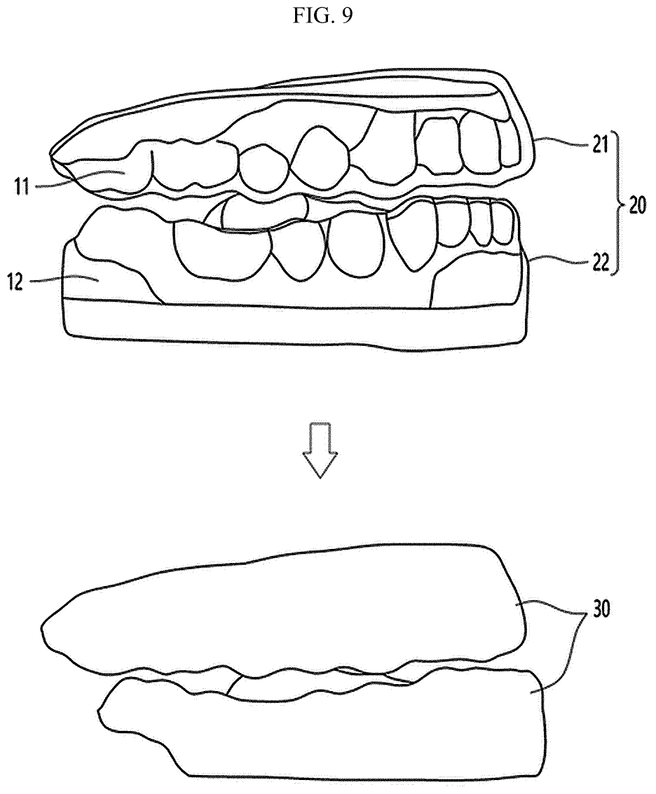

Referring to FIG. 9, the computing device 100 applies the clearance distance corresponding to multiple regions in response to the tooth model 10 including the maxillary model 11 and the mandibular model 12, thereby generating a master model 20 including a first master sub-model 21 and a second master sub-model 22, and may generate basic contour information based on the generated master model 20. Furthermore, the computing device 100 may generate final contour information 30 by adjusting the relative positions between the first master sub-model 21 and the second master sub-model 22 based on the basic contour information (i.e., adjusting the relative position based on the mandibular advancement amount information). In this process, the computing device 100 is designed to accurately fit the oral device to each region by reflecting the user's individual oral structure and the mandibular advancement amount information. The finally generated contour information is utilized as key basic data for the design of the oral device, thereby maximizing the securing of the user's airway and ensuring comfort when the device is worn.

In an embodiment, the computing device 100 may generate the master model 20 by combining the first master sub-model 21 and the second master sub-model 22 while reflecting the mandibular advancement amount information during the process of merging the first and second master sub-models.

In another embodiment, the computing device 100 may generate the tooth model 10 with the positional relationship between the maxilla and the mandible by reflecting the mandibular advancement amount information in the user's tooth model 10. Thereafter, based on the tooth model 10, the computing device 100 may generate and merge the first master sub-model 21 and the second master sub-model 22, thereby generating the master model 20 in which the mandibular advancement amount information is reflected.

According to an embodiment of the present disclosure, the method of manufacturing a customized oral device for sleep disorder improvement using 3D modeling may include generating a plurality of part models based on the contour information (S300).

The generating of the plurality of part models may include generating the first part model corresponding to the maxillary model based on the contour information, and generating the second part model corresponding to the mandibular model based on the contour information.

The contour information (or basic contour information) generated by the computing device 100 does not directly represent the physical thickness or volume of the oral device, but may be composed of linear data defined along the perimeter of the master model. In other words, the contour information does not represent structural elements of the actual oral device, but is a virtual contour used to define the shape of the device, and may serve as a basic reference line for the optimal arrangement of the maxilla and mandible during the design stage.

According to an embodiment, the computing device 100 generates the first part model and the second part model based on the generated contour information. During this process, the contour information acts as a virtual contour applied to each region of the maxillary model and mandibular model, thereby allowing the maxillary and mandibular part models to be accurately defined.

In an embodiment, the computing device 100 may generate the first part model corresponding to the maxillary model based on the contour information. During this step, the appropriate clearance is applied based on the tooth and gum structure of the maxilla, and the clearance plays a key role in determining the shape of the part model. The computing device 100 sets a virtual boundary along the perimeter of the maxillary model based on the contour information and defines the structure of the first part model along this boundary. In this case, each part of the part model is adjusted to the anatomical characteristics of the maxilla and designed to have the optimal thickness and volume by taking into account the securing of the airway and the occlusal condition.

Furthermore, the computing device 100 generates the second part model corresponding to the mandibular model. The process of generating the second part model is also performed based on the contour information. The computing device 100 analyzes the tooth arrangement and gum condition of the mandible and generates the second part model corresponding to the mandible based on the contour information generated along the perimeter of the mandibular model. For the mandible, the advancement information is included, so the shape of the second part model may be adjusted in a specific area to maximize the effect of securing the airway of mandibular advancement. For example, to minimize discomfort that may occur as the mandibular anterior teeth move forward, the thickness and clearance of the corresponding area are adjusted, and thereby the device can be worn stably.

In an embodiment, during the process of generating the first and second part models based on the contour information, the thickness may be provided only outward from the outlines of the maxilla and mandible. The computing device 100 adjusts each part model to an appropriate thickness based on the contour information corresponding to the tooth model, thereby enabling actual manufacturing.

Specifically, the outlines of the maxilla and mandible are theoretically defined as planes with zero thickness. However, for actual manufacturing of the oral device, the outlines should be given the thickness. In this case, thickness is applied only to the outer side. This is to ensure that the oral device is designed to be accurately fitted to the teeth and gums. When the thickness is also applied inwardly based on the contour information, the teeth may not fit properly within the device, making the device unusable. Therefore, to prevent this problem, the computing device 100 generates each part model by applying the thickness only to the outer side of the outline.

The first part model and the second part model generated by the computing device 100 are digital models that reflect the structures of the maxilla and mandible, respectively, and are then integrated to form a single, integrated oral device model.

According to an embodiment, the first part model and the second part model may include at least one of a lingual space for securing an airway, a wing part formed through a shape that covers a tooth, and an open hole formed through a hole shape in one region for pressure distribution.

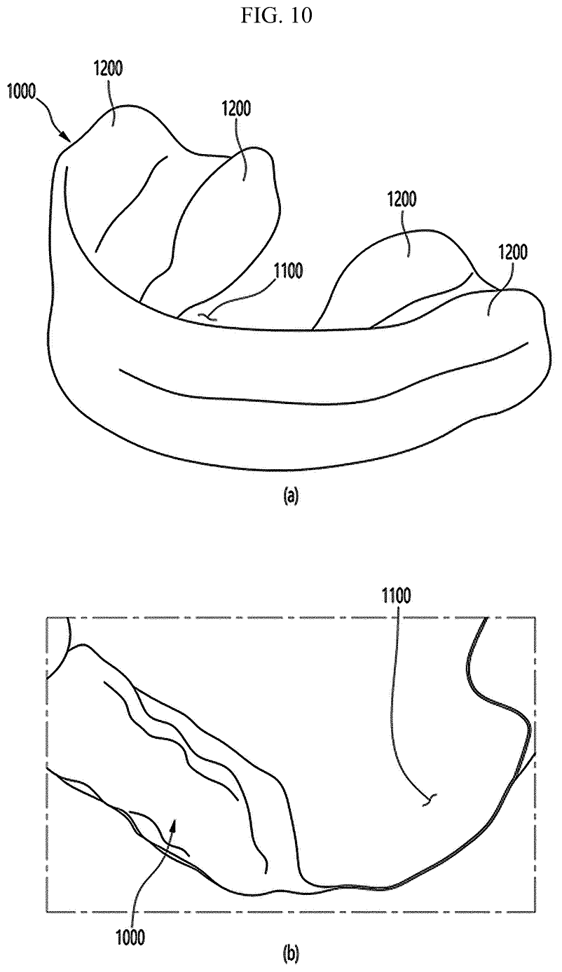

More specifically, referring to (a) of FIG. 10, a wing part 1200 may be formed in a shape that surrounds the teeth on the side of the oral device. The wing part 1200 provides stability to prevent the oral device from dislodging from the teeth during sleep, while also facilitating the device's detachment. The wing part specifically serves to increase the contact area between the maxilla and the mandible and increase the device's fixation force.

The computing device 100 may analyze the user's oral structure and tooth arrangement data to determine the optimal position and size for the wing part. For example, the computing device 100 designs the wing part by considering the area where the user's teeth are distributed and the angles and heights of teeth. The height of the wing part may be designed to be relatively low near the maxillary anterior teeth to facilitate detachment, while the height of the wing part may be designed to be greater near the mandibular molars to enhance stability.

Furthermore, the computing device 100 may form wing parts on both the outer and inner sides of the teeth, which are designed to further enhance the fixation force of the oral device and prevent the device from dislodging from the teeth. The design of these wing parts may be adjusted to fit the individual user's tooth structure and oral condition, minimize any discomfort that may arise during the user's process of inserting and removing the device, and maintain a stable position even during long-term wear.

In an embodiment, the wing parts are provided in the area extending from the maxillary and mandibular anterior teeth to the molars, and the height and shape of the wing parts may be designed differently for each region. The wing parts may be formed on both the inner and outer sides of the teeth, and are designed to simultaneously ensure both device stability and comfort. For example, the inner wing part near the maxillary anterior teeth may be designed with a relatively low height so that the device can be easily detached, thereby allowing the user to easily insert and remove the device. On the other hand, the outer wing part may be designed with a slightly higher height to compensate for potential instability during the detachment in the anterior region.

Near the molars, both the inner and outer wing parts may be designed with relatively high heights to enhance device stability. In the embodiment, molar teeth may be considered to include premolars and molars. Premolars are generally anterior molars that perform a chewing function while experiencing relatively little pressure during occlusion. Molars are posterior molars that primarily function to grind food and may experience significant pressure during occlusion.

The inner wing part firmly holds the sides of the teeth to prevent the device from moving during sleep, while the outer wing part firmly supports the device to prevent the device from moving laterally. This design ensures that the device is securely fixed to the teeth during sleep, while making detachment of the device easy.

Furthermore, referring to (b) of FIG. 10, in the embodiment, a lingual space 1100 may be an empty space formed inside the oral device, particularly in the area where the tongue is positioned. (b) of FIG. 10 is an exemplary diagram of the oral device from below when the oral device is worn. For example, when the user's tongue is pushed by the device due to its thickness when worn, the airway may not be sufficiently secured, making breathing difficult. To prevent this, the lingual space is formed inside the device to allow the tongue to be comfortably positioned. The lingual space is particularly important for securing the airway and is designed to maintain a natural tongue position when the device is worn, facilitating breathing during sleep.

According to an embodiment, the lingual space 1100 may be formed in the posterior region of the maxillary anterior teeth. The computing device 100 may analyze the user's oral structure and tongue position to identify the posterior region of the maxillary anterior teeth and generate the first part model to form the lingual space in the identified region. This allows the tongue to be positioned correctly and the airway to be unobstructed, enabling the user to breathe smoothly while wearing the device.

Furthermore, in an embodiment, the computing device 100 may analyze oral data to determine the location and number of open holes to effectively distribute pressure that may occur while the device is worn. The open holes are intended to alleviate excessive pressure concentrated in specific areas when the oral device is worn, thereby minimizing user discomfort and enhancing device durability. The excessive pressure may overload teeth and gums in a specific area, resulting in pain or discomfort in the long term, which may hinder effective use of the device. Therefore, the computing device 100 may include open holes in the individual part generation process to prevent such problems in advance.

Specifically, the computing device 100 analyzes the user's tooth structure and pressure distribution, and then identifies an area where the excessive pressure is likely to be applied. For example, the area around the mandibular molars is a region where significant pressure may be concentrated during occlusion. Therefore, the computing device 100 may design the opening hole in this area to effectively distribute the pressure. During this process, the computing device 100 calculates the optimal size of the opening hole so that it is neither too large nor too small, thereby maximizing the pressure-distribution effect while maintaining the functionality of the device.

Additionally, some users may have relatively long or protruding crowns on their anterior teeth. In such cases, pressure is likely to be concentrated in the anterior region as well, and thus additional opening holes may be required. The computing device 100 may analyze the user's anterior tooth structure and optimize the size and number of opening holes based on the length and shape of the anterior teeth. For example, for users with long anterior crowns, multiple open holes are designed on the lingual or mesio-distal surfaces of the anterior teeth to disperse excessive pressure applied to these areas. In this way, even when the device is worn for a prolonged period of time, it is possible to reduce the burden applied to the anterior teeth and improve the wearing comfort of the device.

When determining the locations of the open holes, the computing device 100 considers the interaction between the area where pressure is concentrated and the surrounding structures. For example, when the contact with the maxilla occurs strongly near the mandibular molars, multiple open holes may be arranged in this area to evenly distribute the pressure. In this case, the computing device 100 may also optimize the sizes and shapes of the open holes to minimize discomfort that may occur during long-term wear.

Furthermore, the open holes may be appropriately designed for users with long or protruding anterior teeth. The computing device 100 arranges multiple open holes of appropriate sizes according to the lengths of the anterior teeth, thereby effectively distributing pressure that may be concentrated on the anterior teeth. This allows the oral device to smoothly fit the user's anterior teeth, thereby minimizing discomfort even during long-term wear.

Furthermore, the computing device 100 may adjust the size and number of each opening hole to fit the user's oral structure. For example, larger opening holes may be designed for areas subject to greater pressure, while smaller opening holes may be designed for areas subject to less pressure, thereby optimizing pressure distribution. This increases the durability of the device and allows the user to wear the device more comfortably.

Based on the analysis results, the computing device 100 generates first and second part models, each of which includes opening holes optimized for pressure distribution. The open hole designed near the anterior region is also designed based on the above-described analysis, and it is particularly useful in cases where the anterior region is relatively long or pressure concentration is expected. These opening holes reduce potential discomfort during device wear and ensure a stable fit of the device to the user's oral structure, thereby maximizing long-term therapeutic effectiveness.

The design of these open holes not only enhances the functionality of the device, but also plays a key role in helping maintain the user's oral health over the long term. Therefore, the computing device 100 provides an optimal design that considers both user comfort and device effectiveness by precisely adjusting the location, size, and number of open holes

According to various embodiments, the computing device 100 may identify the molar area based on the user's oral data and design the oral device model that prevents bruxism and clenching. Specifically, the computing device 100 analyzes various oral data, such as the user's occlusal force, tooth structure, and jaw movement, to precisely determine the distribution of forces occurring in the molar area and the occlusal condition. Based on this data, the computing device 100 may design an empty space to prevent the molars from contacting each other, thereby preventing force from being applied to the molars during occlusion.

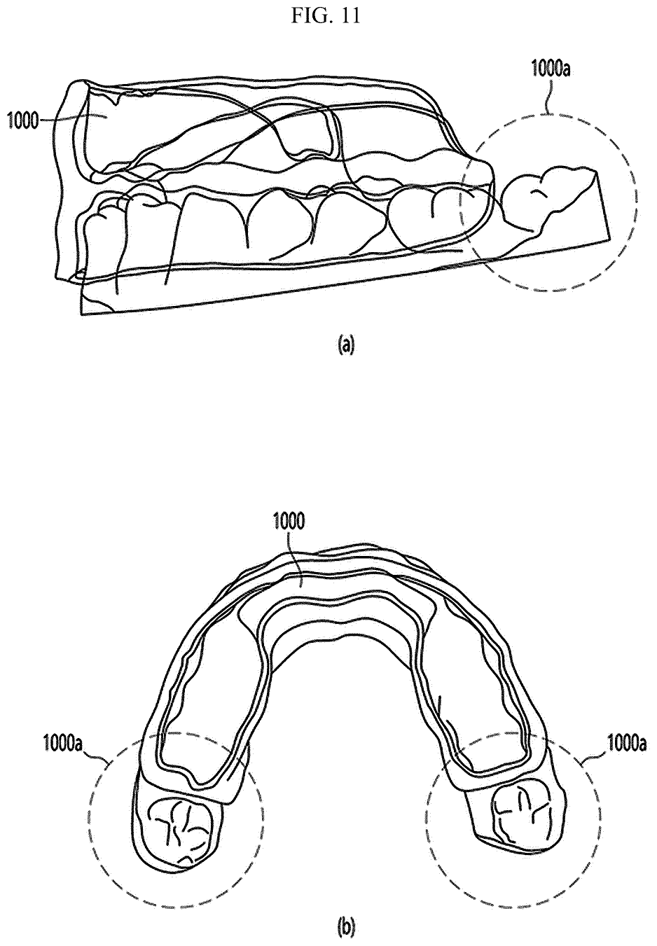

Referring to (a) and (b) of FIG. 11, a molar area 1000a is a region where bruxism and clenching primarily occur. The computing device 100 generates an empty space in this area to prevent the molars from directly contacting each other during occlusion. This design effectively blocks pressure applied to the molars, thereby playing a key role in preventing the user from unintentionally clenching or grinding their teeth.

According to an embodiment, the molar area 1000a of the present disclosure may primarily refer to the area related to molars. The molars are teeth located posterior on the maxilla and mandible, are larger than premolars, and experience greater pressure during occlusion. While the molars play a key role in grinding food, the molars are also a region where unnecessary pressure, such as bruxism or clenching, is primarily concentrated. The present disclosure provides a function that effectively prevents excessive pressure that may occur during occlusion by treating the molar region as a void, thereby preventing direct contact between the molars when the device is worn.

According to an embodiment, as illustrated in FIG. 11, the oral device model of the present disclosure may be manufactured to cover the molars up to the premolars, leaving the molars uncovered or covering only the anterior portion of the molars. This design approach may be adopted to block the strong pressure that may occur in the molar region while maintaining the stability of the device. Therefore, by maintaining proper occlusion in the premolar region while preventing occlusion in the molar region, the device may prevent bruxism or clenching when the device is worn, thereby protecting the user's oral health.

To design the void space in the molar region, the computing device 100 first identifies the locations of the molars and the contact pattern between the teeth based on the user's oral data. During this process, the computing device 100 precisely analyzes the area where occlusal forces are concentrated, thereby dispersing force that may occur during occlusion and adjusting the molar region to prevent occlusion. This may prevent unnecessary pressure from being applied to the molars even during sleep, and prevent tooth damage caused by bruxism or clenching.

Furthermore, this void space design is not simply asymmetrical, and is precisely designed by considering the user's jaw movement data and the occlusal condition of the maxilla and mandible. The computing device 100 analyzes the user's jaw movement image data to determine the optimal void size and location to minimize the possibility of contact in the molar region. This prevents the user from experiencing discomfort when wearing the device even if the void is formed, and maintains comfort even during long-term wear.

Consequently, the computing device 100 provides the customized oral device that prevents tooth grinding and clenching through this design process, and helps the user wear the device stably even during sleep. This may maximize both the wearing comfort and functionality of the oral device, thereby providing the user with a more comfortable and effective sleep environment.

According to an embodiment of the present disclosure, the method of manufacturing a customized oral device for sleep disorder improvement using 3D modeling may include generating an oral device model based on a plurality of part models (S400).

In a specific embodiment, the generating of the oral device model may include generating an integrated model by connecting the plurality of part models into one, performing surface processing on the integrated model, and generating the oral device model by performing inner surface machining on the surface-processed integrated model based on the contour information.

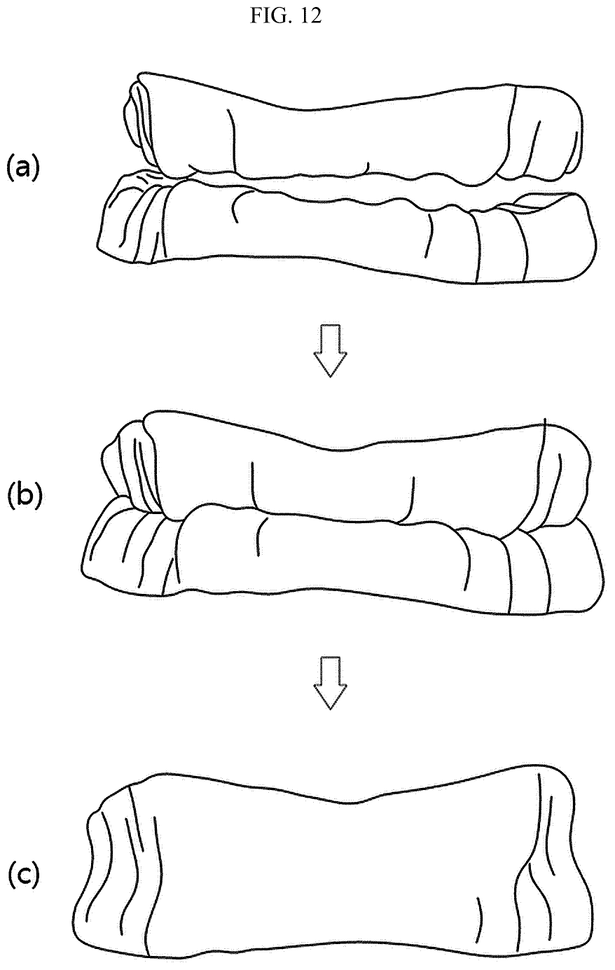

Specifically, the generating of the oral device model begins by connecting a plurality of part models into one to generate an integrated model. In this case, the part models correspond to the maxilla and mandible, as illustrated in (a) of FIG. 12, and are designed to be optimized for the user's oral structure. During the process of merging the part models, the space between the part models is filled, as illustrated in (b) of FIG. 12. Specifically, during the process of merging the part models, new material is added to the boundaries of each model, or the existing models are pressed against each other, thereby filling the space without visible seams and transforming them into a single, integrated structure.

Subsequently, as illustrated in (c) of FIG. 12, the surface processing is performed to ensure that the shapes of the models are seamlessly connected. This surface processing process may be performed using wrapping or smoothing techniques.

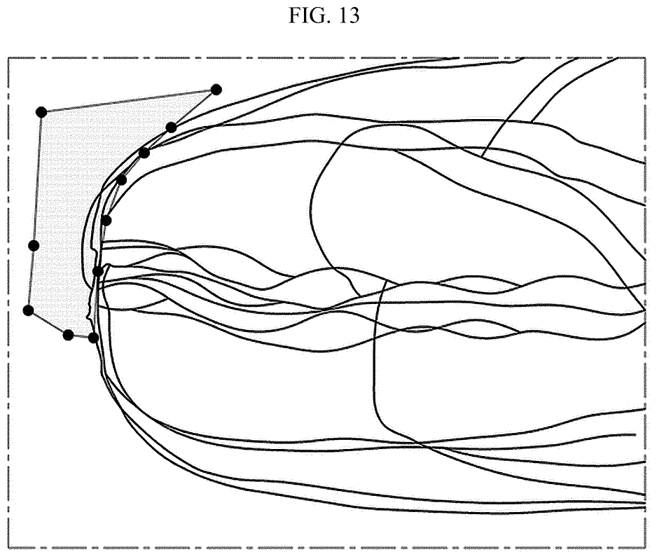

The computing device 100 may apply the surface processing techniques such as wrapping or smoothing to make the appearance of the integrated model more natural and smooth. These techniques smoothly connect the boundaries between the part models, ensuring the seamless appearance for the integrated model while simultaneously enhancing the wearing comfort of the device. For example, as illustrated in FIG. 13, the computing device 100 applies the wrapping technique to each corner and curved portion of the integrated model to naturally connect the boundaries, thereby smoothing the surface of the integrated model. This may minimize any foreign body sensation when the oral device is worn, and allow the oral device to be worn smoothly within the oral cavity.

That is, the computing device 100 may perform surface processing to generate an integrated model (i.e., a surface-processed integrated model) in which the boundaries between individual part models are smoothly connected.

Meanwhile, during the process of merging the part models and applying surface processing (e.g., lapping and smoothing), the shape of the integrated model becomes slightly thicker than the original model. This is because the overall volume increases as the perimeters of the individual part models are smoothly connected. This may cause the problem that the internal space of the integrated model, particularly the area where the teeth are inserted, is narrowed.

To address this problem, after the surface processing of the integrated model is completed, the computing device 100 performs inner surface machining based on the contour information (i.e., the contour information generated based on the master model) to generate the oral device model.

Specifically, the computing device 100 utilizes the contour information to precisely subtract and reconstruct the inner surface of the oral device from the 3D model. During this process, the computing device 100 re-secures the internal space narrowed by the surface processing, thereby generating an optimal space where teeth may be accurately inserted. The contour information generated based on the master model accurately reflects the anatomical structure of the teeth and serves as a guide for precisely removing the inner surface of the device based on the detailed contours of the teeth and gums.

Through this process, the computing device 100 removes unnecessary protrusions or excessively narrow spaces within the oral device and optimizes the internal structure to ensure the precise engagement of the device with the user's teeth and gums. The contour information in which the clearance distance and the mandibular advancement amount are reflected ensures that pressure applied to the teeth and gums is appropriately distributed when the device is worn, thereby ensuring optimal wearing comfort and functionality of the oral device.

Consequently, the precise inner surface machining performed by the computing device 100 ensures that the oral device adheres precisely to the user's teeth and gums while remaining comfortable to wear, maximizing both the wearing comfort and functionality of the device. In this way, the completed oral device model allows the user to maintain comfort even during long-term wear, effectively secures the airway during sleep, and provides a stable occlusal condition.

Since the oral device model of the present disclosure is integrally implemented, separate accessories are not required to connect the maxillary and mandibular portions. This simplifies the device structure, making hygiene management easier and reducing the burden on the user in maintaining the device's cleanliness. Furthermore, the integrated structure reduces the risk of device breakage and ensures stability even during long-term use.

In particular, the oral device model of the present disclosure includes the clearance designed to reflect the anatomical characteristics of each tooth and gum, thereby effectively dispersing pressure generated during wear. This prevents pressure from being concentrated in a specific area and minimizes discomfort during long-term wear. Furthermore, the clearance design allows the device to move within a small range relative to the user's teeth and gums, helping to maintain a natural occlusion while minimizing strain on the temporomandibular joint. This prevents injuries that may occur during device use and further enhances overall wearing comfort.

Consequently, the oral device model of the present disclosure is hygienic and easy to maintain due to its integrated structure without connecting components between the maxilla and the mandible, and provides the pressure distribution and freedom of movement through its clearance design to allow comfort and safety to be maintained even during long-term use, thereby securing the airway during sleep and effectively providing the stable occlusal condition.

According to an embodiment of the present disclosure, the method of manufacturing a customized oral device for sleep disorder improvement using 3D modeling may include processing the tooth model as an input to a deep learning model to generate the oral device model.

The deep learning model may be a neural network model pre-trained using a training data set composed of tooth models and oral device models of multiple users.

Specifically, the computing device 100 may construct the training data set including the maxillary and mandibular tooth models collected from multiple users and the oral device model customized for each user.

The training data set may include a training input data set related to the tooth models of multiple users and a training output data set related to the oral device models corresponding to each tooth model.

The training input data includes data related to the tooth model generated based on oral data such as the user's tooth structure, arrangement, gum condition, and jaw movement, and is provided as an input to the deep learning model. The training output data includes data for the oral device model customized for each user and is used as correct data for comparison with the oral device model predicted by the deep learning model.

For example, the computing device 100 provides a specific user's maxillary and mandibular tooth models as input data, and the deep learning model predicts the oral device model suitable for the user based on the input data. The predicted oral device model is compared with the actually used correct oral device model, and in this process, an error between the predicted result and the correct data is derived. The derived error is used to adjust the connection weights within the deep learning model through backpropagation. The deep learning model is repeatedly trained to minimize this error, thereby improving its ability to generate an optimal oral device model corresponding to each tooth model. Through this training process, the deep learning model develops into a neural network model that may automatically generate the customized oral device model that optimizes airway securing and wearing comfort by receiving a new user's teeth model as an input.

That is, the computing device 100 utilizes the deep learning model trained from the tooth models and oral device models collected from multiple users to automatically generate a customized oral device model for a new user after receiving the user's tooth model. During this process, the computing device 100 predicts the oral device model based on the input tooth model, and the deep learning model designs the device for optimizing the securing of the airway and the wearing comfort based on the trained data. The oral device model predicted by the deep learning model is generated by reflecting the optimal thickness, clearance distance, and mandibular advancement amount tailored to each user's oral structure and characteristics, thereby helping the device effectively improve sleep disorders.

In this way, by generating the deep learning model and automatically generating the customized oral device model based on a new user's tooth model, a device optimized for the user's oral structure and characteristics may be quickly and efficiently provided. This reduces the time required to design the customized oral device, while simultaneously maximizing accuracy and comfort, resulting in a more effective device for improving sleep disorders.

In various embodiments, the computing device 100 may further improve the output accuracy of the final deep learning model by including the jaw movement image data in addition to the tooth model as the training input data during training. Specifically, the jaw movement image data provides valuable information for comprehensively analyzing how the user's jaw moves during sleep, including patterns such as forward and backward movement, left and right rotation, and up and down movement. By including the jaw movement data in the training input data, the deep learning model may more precisely train, along with the tooth model, how the user's mandible contributes to widening the airway and how stable the contact between the maxilla and mandible is during occlusion.

The computing device 100 utilizes the jaw movement image data and tooth model data collected from multiple users as the training input data for the neural network model, enabling the neural network model to predict various movements that may occur while each user wears the oral device. For example, when a particular user tends to have a mandible that retracts or advances during sleep, the deep learning model may train this and optimize the mandibular advancement amount to ensure the stable airway while the oral device is worn. This training process reflects dynamic data in the deep learning model that may not be obtained from static tooth arrangement information alone, thereby generating a more accurate and personalized oral device model.

By including the jaw movement image data in the training input data, the deep learning model designs the optimal oral device model by considering the user's oral structure and mandibular movement patterns from a more comprehensive perspective. The output of the deep learning model predicts not only the interaction between the tooth model and the oral device model, but also the device's suitability based on the changes in jaw movement and occlusion, thereby enhancing the output accuracy of the user-customized oral device model. Consequently, the finally generated oral device model provides more precise wearing comfort and is designed to comfortably and effectively secure the user's airway even during sleep.

Furthermore, according to an embodiment, the computing device 100 may include a function for simulating the performance of the oral device model. It is possible to virtually simulate and predict the extent to which the oral device model generated by the deep learning model may contribute to securing the airway during actual use, and whether it imposes any burden on the user's temporomandibular joint. The computing device 100 performs the simulation based on the user's oral structure, tooth arrangement, and jaw movement data, and may predict how the optimized oral device will widen the airway and distribute pressure when worn.

These simulation results are fed back as the training data for the deep learning model, contributing to the generation of the more accurate and precise model for the next oral device model design. Consequently, the computing device 100 provides the optimized oral device model that reflects the user's individual oral characteristics and movements during sleep, and designs the device so that the user may wear the optimized oral device model comfortably for long periods of time.

In various embodiments, the computing device 100 may perform the oral device design process utilizing the pre-trained artificial intelligence model.

In an embodiment, the computing device 100 may input the user's tooth model 10 into the pre-trained artificial intelligence model and acquire the user-customized oral device model from the output of the artificial intelligence model.

In various embodiments, the user's tooth model 10 may reflect the user's mandibular advancement amount information, thereby applying the positional relationship between the maxilla and mandible, but is not limited thereto.

For example, the computing device 100 may convert a 3D mesh of a user's tooth model into voxels of a preset size. The preset size may be set to an appropriate size considering the computational load when used as training data for an AI model, but the specific size is not limited.

For example, the computing device 100 may input dental voxels into a pre-trained AI model (e.g., a UNet-based architecture) and acquire oral appliance voxels as the output of the AI model.

The computing device 100 may convert the output oral appliance voxels into a 3D mesh of the oral appliance using a marching cube algorithm, but the specific algorithm is not limited thereto.

In an embodiment, the computing device 100 may acquire the master model 20 generated in response to the user's tooth model 10.

In various embodiments, the master model 20 may be generated using the methods described herein, but is not limited thereto. As another example, the computing device 100 may acquire information on a pre-designated vertex group for a specified area where clearance should be provided between the maxilla and the mandible with respect to the user's tooth model 10.

The information regarding the vertex group may be manually designated by the user (e.g., using a mouse drag input), but may also be automatically designated using a pre-trained segmentation AI model, according to the embodiment.

The computing device 100 may generate the master model by providing the clearance to the designated vertex group.

For example, the computing device 100 may add the thickness to the entire tooth model 10 mesh according to a preset rule, and may add the thickness to each vertex group designated for each tooth region according to the preset rule.

Thereafter, the computing device 100 may perform processing to transform the surface of the mesh with added thickness into a smooth shape. For example, the computing device 100 may replicate the original mesh and perform processing (e.g., remesh, fatten, subdivision) to transform the surface of the replicated mesh into the smooth shape. The computing device 100 may attach the processed replicated mesh to the original mesh (e.g., naturally attaching the processed replicated mesh to the original mesh through shrinkwrap). According to an embodiment, the computing device 100 may additionally perform the same process to generate the master model 20 having a smooth and natural shape.

In an embodiment, the computing device 100 may generate the first master sub-model 21 and the second master sub-model 22 for each of the maxilla and mandible using the above processes.

In various embodiments, the computing device 100 may secure the undercut space necessary for the removal of the oral device by replicating the mesh for the mandible multiple times during the process of generating the second master sub-model 22 for the mandible, moving n replicated meshes by different intervals (e.g., −1 to −n) in the z-axis direction, and then merging the replicated meshes, but is not limited thereto.

The computing device 100 may generate the master model 20 by merging the first master sub-model 21 and the second master sub-model 22.

In an embodiment, the computing device 100 may input the user's tooth model 10 to the pre-trained artificial intelligence model and acquire the master model 20 from the output of the artificial intelligence model.

In an embodiment, the computing device 100 may generate the final oral device model by performing a subtraction operation on the master model 20 from the oral device model generated using the artificial intelligence model.

Through this, the computing device 100 may generate the user-customized oral device by correcting the oral device model generated using the artificial intelligence model using the master model 20. According to the embodiment, additional corrections may be performed on the generated customized oral device, but the present disclosure is not limited thereto.

According to an embodiment of the present disclosure, a manufacturing system for a customized oral device for sleep disorder improvement through 3D modeling may include an output device that outputs and manufactures the oral device model. The output device may be a high-precision output device, such as a 3D printer, and serves to physically manufacture the user-customized oral device model. For example, the output device may include various 3D printing methods, such as stereolithography (SLA), selective laser sintering (SLS), and fused deposition modeling (FDM), but is not limited thereto. The output device may precisely output the complex shapes and fine structures of the oral device model, allowing the digital oral device model generated during the design stage to be implemented as a physical product.

In an embodiment, the output device may be used as a device for outputting an integrated oral device model including maxillary and mandibular part models. More specifically, when the 3D oral device model generated by the computing device 100 is transmitted to the output device, the output device outputs the oral device model layer by layer using high-resolution 3D printing technology. The output oral device is precisely manufactured according to the tooth structure and anatomical characteristics of the gums by taking into account the interdigitation of the maxilla and mandible.

That is, the output device outputs the customized oral device in the form optimized for the individual user's teeth and gum structure. The customized oral device is an oral device completed through subsequent processes, and may be used for therapeutic purposes aimed at sleep disorder improvement. In an embodiment, the subsequent processing may include a polishing process to smooth the surface of the device after physical manufacturing. Furthermore, the subsequent process may include cleaning and inspection processes following the polishing process. This series of processes may contribute to maximizing the precision and effect of the customized oral device and effectively improving the user's sleep disorder.

Throughout this specification, “computational model,” “neural network” and “network function” may be used with the same meaning. (Hereinafter, it shall be described consistently as a neural network.) A data structure may include a neural network. Furthermore, the data structure including the neural network may be stored on a computer-readable medium. The data structure including the neural network may also include data input to the neural network, weights of the neural network, hyperparameters of the neural network, data obtained from the neural network, activation functions associated with each node or layer of the neural network, and a loss function for training the neural network. The data structure including the neural network may include any of the components described above. That is, the data structure including the neural network may be configured to include all or any combination of the data input to the neural network, the weights of the neural network, the hyperparameters of the neural network, the data obtained from the neural network, the activation functions associated with each node or layer of the neural network, and the loss function for training the neural network, etc. In addition to the above-described components, the data structure including the neural network may include any other information that determines the characteristics of the neural network. Furthermore, the data structure may include any form of data used or generated during the computational process of the neural network, and is not limited to the above-described matters. The computer-readable medium may include a computer-readable recording medium and/or a computer-readable transmission medium. The neural network may be composed of a set of interconnected computational units which may generally be referred to as nodes. These “nodes” may also be referred to as neurons. The neural network is configured to include at least one node.

Operations of the method or algorithm described with reference to the embodiment of the present disclosure may be directly implemented in hardware, in software modules executed by hardware, or in a combination thereof. The software module may reside in a random access memory (RAM), a read only memory (ROM), an erasable programmable ROM (EPROM), an electrically erasable programmable ROM (EEPROM), a flash memory, a hard disk, a removable disk, a compact disc read-only memory (CD-ROM), or in any form of computer-readable recording medium known in the art to which the invention pertains.

The components of the present disclosure may be embodied as a program (or application) and stored in a medium for execution in combination with a computer which is hardware. The components of the present disclosure may be executed in software programming or software elements, and similarly, embodiments may be realized in a programming or scripting language such as C, C++, Java, and assembler, including various algorithms implemented in a combination of data structures, processes, routines, or other programming constructions. Functional aspects may be implemented in algorithms executed on one or more processors.

Those skilled in the art to which the present disclosure pertains will appreciate that the various exemplary logical blocks, modules, processors, means, circuits, and algorithm steps described in connection with the embodiments disclosed herein may be implemented as electronic hardware, various forms of programs or design codes (for convenience, referred to as software herein), or a combination of both. To clearly illustrate this interchangeability of hardware and software, various exemplary components, blocks, modules, circuits, and steps have been generally described above in terms of their functionality. Whether such functionality is implemented as hardware or software depends upon particular applications and design constraints imposed on the overall system. Those skilled in the art to which the present disclosure pertains may implement the described functionality in varying ways for each particular application, but such implementation decisions should not be interpreted as causing a departure from the scope of the present disclosure.

Various embodiments described herein may be implemented as methods, apparatuses, or manufactured articles using standard programming and/or engineering techniques. The term “manufactured article” includes computer programs, carriers, or media accessible from any computer-readable device. For example, the computer-readable media include, but are not limited to, magnetic storage devices (e.g., hard disks, floppy disks, magnetic strips, etc.), optical discs (e.g., CDs, DVDs, etc.), smart cards, and flash memory devices (e.g., EEPROMs, cards, sticks, key drives, etc.). In addition, various storage media presented herein include one or more devices and/or other machine-readable media for storing information. The term “machine-readable media” includes, but is not limited to, wireless channels and various other media capable of storing, retaining, and/or transmitting command(s) and/or data.

It is to be understood that the specific order or hierarchical structure of steps in the presented processes is merely an example of exemplary approaches. It is to be understood that the specific order or hierarchical structure of steps in the processes may be rearranged based on design priorities within the scope of the present disclosure. The accompanying method claims present elements of various steps in a sample order, but are not intended to be limited to the specific order or hierarchical structure presented.

According to various embodiments of the present disclosure, by providing the method through which the user-customized oral device can be efficiently and precisely designed and manufactured using 3D modeling, it is possible to maximize the effect of the sleep disorder improvement. This may allow users to experience more comfortable wearing and optimize the therapeutic effectiveness of the oral device.

In addition, according to the present disclosure, by accurately reflecting the oral structures and tooth conditions of individual users during the design process of the oral device, it is possible to overcome the inconveniences and limitations of the existing standardized devices. This allows for the provision of the customized devices suitable for various users, thereby increasing the accessibility of sleep disorder treatment.

Additionally, according to the present disclosure, by automating the generation process of the oral device model using the deep learning model, it is possible to reduce the manufacturing costs and time and implement more efficient design and production. This may popularize customized devices and provide more people with the opportunity to overcome sleep disorders.

The effects of the present disclosure are not limited to the above-described effects, and other effects that are not mentioned may be obviously understood by those skilled in the art from the following description.

The description of the present embodiments is provided to enable those skilled in the art to make or use the present disclosure. Various modifications to these embodiments will be readily apparent to those skilled in the art, and the generic principles defined herein may be applied to other embodiments without departing from the scope of the present disclosure. Therefore, the present disclosure is not limited to the embodiments set forth herein, but is to be construed in the broadest scope consistent with the principles and novel features disclosed herein.

What is claimed is:

1. A method of manufacturing a customized oral device for sleep disorder improvement using 3D modeling, performed on one or more processors of a computing device, the method comprising:
generating a tooth model including a maxillary model and a mandibular model;
generating contour information based on the tooth model;
generating a plurality of part models based on the contour information; and
generating an oral device model by integrating the plurality of part models.

2. The method of claim 1, wherein the generating of the contour information includes:
generating a master model by applying a clearance distance corresponding to each of multiple regions of the tooth model;
generating basic contour information based on the master model; and
generating the contour information by correcting the master model based on the basic contour information.

3. The method of claim 2, wherein the clearance distance is set to allow for pressure distribution and a predetermined amount of movement, and determined differently for each region based on anatomical characteristics of a tooth, interaction with a surrounding structure, and the physical characteristics of an individual tooth, and
the generating of the basic contour information includes:
identifying multiple predefined regions in the tooth model;
calculating the clearance distance for each region corresponding to each of the multiple regions;
applying the calculated clearance distance for each region to each of the multiple regions; and
performing surface processing corresponding to the region to which the clearance distance is applied.

4. The method of claim 2, wherein the generating of the contour information includes:
generating mandibular advancement amount information based on oral data; and
generating the contour information by adjusting a relative position between a maxilla and a mandible of the master model based on the generated mandibular advancement amount information, and
the oral data includes information regarding a user's oral condition and includes impression acquisition result data and jaw movement image data.

5. The method of claim 1, wherein the generating of the plurality of part models includes:
generating a first part model corresponding to the maxillary model based on the contour information; and
generating a second part model corresponding to the mandibular model based on the contour information.

6. The method of claim 5, wherein the first part model and the second part model include at least one of a lingual space for securing an airway, a wing part formed through a shape that covers a tooth, and an open hole formed through a hole shape in one region for pressure distribution.

7. The method of claim 1, wherein the generating of the oral device model includes:
generating an integrated model by connecting the plurality of part models into one;
performing surface processing on the integrated model; and
generating the oral device model by performing inner surface machining on the surface-processed integrated model based on the contour information.

8. The method of claim 1, further comprising generating the oral device model by processing the tooth model as an input to a deep learning model,
wherein the deep learning model is a neural network model pre-trained using a training data set composed of tooth models and oral device models of multiple users.

9. The method of claim 1, further comprising generating an oral device model by outputting the generated oral device model.

10. A computing device for performing the method of claim 1, comprising:
a memory storing one or more instructions; and
a processor configured to execute the one or more instructions stored in the memory,
wherein the processor executes the one or more instructions to perform the method of claim 1.

11. A non-transitory computer-readable recording medium storing a computer program to perform the method of claim 1 when connected to a computing device which is hardware.

12. A system for manufacturing a customized oral device for sleep disorder improvement using 3D modeling, the system comprising:
a computing device generating an oral device model; and
an output device outputting and manufacturing the oral device model,
wherein the computing device generates a tooth model including a maxillary model and a mandibular model,
generates contour information based on the tooth model,
generates a plurality of part models based on the contour information, and
generates the oral device model based on the plurality of part models.
